(19) United States
(12) Patent Application Publication
(10) Pub. No.: US 2008/0285707 A1
(43) Pub. Date: Nov. 20, 2008

(54) System and Method for Medical Navigation

(75) Inventors: Willi A. Kalender, Mohrendorf (DE); Ralf Petzold, Erlangen (DE)

Correspondence Address: LERNER GREENBERG STEMER LLP, P O BOX 2480, HOLLYWOOD, FL, 33022-2480, US

(73) Assignee: CAS INNOVATIONS AG, Erlangen (DE)

(21) Appl. No.: 12/091,386

(22) PCT Filed: Oct. 23, 2006
(86) PCT No.: PCT/EP06/10178

§ 371 Date: Jun. 6, 2008

(30) Foreign Application Priority Data
Oct. 24, 2005 (DE) 10 2005 051 102.3

(51) Int. Cl. A61B 6/00

(52) U.S. Cl. 378/11

(57) ABSTRACT

The invention relates to a system and a method for medical navigation. In order to reduce the time required for medical navigation for transcutaneous interventions, the invention proposes that the position of a medical instrument (5) in an object (4) be determined with the aid of projection images (13, 14), and that the position be indicated in a three-dimensional structural image (11). The invention can be carried out in particular with C-arc X-ray appliances and CT X-ray appliances.

The invention relates to a system and a method for navigation of instruments in medicine (medical navigation). The invention also relates to a computer program for medical navigation. In particular, the invention can be used for transcutaneous interventions.

Image-guided interventions, in particular CT-guided interventions, are nowadays part of clinical routine. In contrast to an invasive surgical operation, minimal-invasive image-guided interventions in this case make it possible for a doctor to work with minimal injuries to the patient. This on the one hand reduces the clinical costs. It also reduces the risk of complications, and has a positive cosmetic effect.

The accuracy and the speed with which a medical instrument such as a puncture needle or a reamer is placed in the patient's body depend to a major extent on the skill of the radiologist. In particular, a procedure such as this requires a large amount of experience. A multiplicity of monitoring scans are frequently required in order to determine the exact position of the instrument and if necessary to correct it, until the instrument is located at the desired destination point. This is particularly necessary in those applications in which incorrect positioning of the instrument can lead to life-threatening patient states. The frequent monitoring scans not only lengthen the duration of the intervention but also increase the radiation dose for the patient.

One object of the present invention is to reduce the time required for medical navigation for transcutaneous interventions.

This object is achieved by a system for medical navigation according to Claim 1. According to the invention, the system has: means for creation of a three-dimensional structural image of an object, means for creation of at least two projection images of a medical instrument within the object from different angles, and an apparatus for representation of the position, as defined by the projection images, of the medical instrument in the three-dimensional structural image.

Furthermore, this object is achieved by a method for medical navigation according to Claim 10. According to this inventive claim, the following steps are provided: creation of a three-dimensional structural image of an object, creation of at least two projection images of a medical instrument within the object from different angles, and representation of the position, as defined by the projection images, of the medical instrument in the three-dimensional structural image.

Furthermore, this object is achieved by a computer program for medical navigation according to Claim 12. This claim provides for the computer program to have: computer program instructions for representation of a position of a medical instrument in a three-dimensional structural image of an object when the computer program is executed on a computer, with the position of the medical instrument being defined by means of at least two projection images created from different angles.

A three-dimensional structural image is a 3D image which images the three-dimensional structure of an object to be examined. This can be done, for example, by a tomographic method, such as computed tomography (CT), in which the object is represented in a series of parallel slice images. In this case, each picture element corresponds uniquely to one point in the recorded object. In other words, there is no super-imposition in the representation.

A projection image is a shadow image as is used in a projection method, for example in a traditional X-ray examination. In this case, the structures of the object are superimposed if they are located one behind the other in the beam path (2D image).

A medical instrument is any apparatus which is suitable for intervention. This includes, in particular, instruments in the relatively narrow sense, such as puncture needles or the like, and instruments in the wider sense, for example implants, aids and the like.

The invention is based on the idea of not using (additional) complex three-dimensional structural images, but projection images which can be created quickly and easily, for determining the position of a medical instrument in the object. Furthermore, the already existing three-dimensional structural image is used in order to indicate the position of the instrument in a simple form. In other words, one fundamental idea of the invention is to combine a previously recorded three-dimensional structural image with information about the current position of the medical instrument, as defined by projection images. The position of the instrument is therefore indicated in the three-dimensional structural image.

The present invention allows simple navigation during transcutaneous interventions. Errors in guidance and positioning of medical instruments can be prevented or identified early. In comparison to other solutions, the invention is distinguished in particular in that it avoids time-consuming monitoring recordings of 3D structural images. Another advantage is that the radiation load on the object is reduced when using X-ray methods.

A further advantage of the invention is that no positioning markings (markers) or the like need be fitted at all to the medical instrument. This simplifies instrument handling. The method can be used with all conventional instruments. However, it is, of course, possible to use markers such as these, for example when the aim is to use the invention in conjunction with other navigation methods.

Advantageous embodiments of the invention are specified in the dependent claims.

In principle, various imaging methods can be used with the invention. A combination of different imaging methods is also possible. However, it is particularly advantageous for the means for creation of the three-dimensional structural image of the object and/or the means for creation of the at least two projection images to comprise an X-ray appliance, so that an X-ray method is used for imaging. This results in the use of an imaging method which is particularly powerful and can be used universally. The X-ray appliance is preferably used to create both types of image (structural image and projection image). However, the three-dimensional structural image in particular can also be created by some other imaging method, for example by magnetic resonance imaging or the like.

In this case, a C-arc X-ray appliance is used as the X-ray appliance in one embodiment of the invention. X-ray appliances such as these are distinguished inter alia by their costs being lower than those of conventional CT X-ray appliances, and by their simple handling. In addition to conventional C-arc X-ray appliances which are equipped with one X-ray source, by means of which the at least two projection images are recorded using a rotating operating method, the use of a C-arc X-ray appliance with at least two X-ray sources (biplanar arrangement) is advantageous. This is because this allows simultaneous operation, that is to say simultaneous recording of a plurality of projection images, thus reducing the time required to determine the position of the medical instrument. The processes for determining the position of the instrument (monitoring scans) can therefore also be carried out in real time and continuously. In this case, continuous monitoring scans are, in particular, recordings made at regular intervals with pulsed radiation (and therefore with a low radiation load) or continuous recordings with constant radiation.

In a further embodiment of the invention, a CT X-ray appliance is used as the X-ray appliance. In addition to conventional CT X-ray appliances which are equipped with one X-ray source, and by means of which the at least two projection images are recorded using a rotating operating method, the use of a CT X-ray appliance with at least two X-ray sources is also particularly advantageous in this case. A biplanar arrangement such as this also allows the CT X-ray appliance to be operated simultaneously. In other words, an examination can be carried out on a plurality of planes at the same time by the interconnection of a plurality of X-ray sources. One advantage in this case is that the time required to determine the position of the medical instrument is once again considerably reduced, allowing real-time monitoring scans and continuous monitoring to be carried out.

Both when using a C-arc X-ray appliance and when using a CT X-ray appliance, the X-ray appliances, if they have only a single X-ray source (rotating operation), are preferably designed such that the interval between two individual recordings is less than one second. This allows the projection recordings to be made very quickly, thus allowing the position of the medical instrument to be determined very quickly. This leads to a reduced examination load on the object to be examined.

In principle, it is possible, for example, for the three-dimensional structural image of the object to be created using a CT X-ray appliance, and for the projection images to be created using a C-arc X-ray appliance. However, one and the same X-ray appliance is preferably used for both types of image (structural image and projection image). This makes it possible to avoid the need to transport patients between different appliances. This is possible since, on the one hand, CT X-ray appliances can create not only three-dimensional structural images but also conventional projection images (2D recordings), and, on the other hand, C-arc X-ray appliances can also be designed such that they can also create CT recordings (3D recordings) in addition to projection images.

A further embodiment of the system according to the invention comprises a data processing unit with a number of functional modules, with each functional module being designed to carry out one specific function or a number of specific functions, according to the described method. In particular, the apparatus for representation of the position, as defined by the projection images, of the medical instrument in the three-dimensional structural image is in the form of a functional module such as this.

The functional modules may be hardware modules or software modules. In other words, the invention, to the extent that it relates to the data processing unit, may be implemented either in the form of computer hardware in the form of computer software, or in the form of a combination of hardware and software. If the invention is implemented in the form of software, the system functionality described here is implemented by computer program instructions when the computer program is executed on a computer.

In this case, the computer program instructions are implemented in a manner known per se in any desired programming language, and may be provided in any desired form for the data processing unit, for example in the form of data packets which are transmitted via a computer network, or in the form of a computer program product which is stored on a floppy disk, a CD-ROM or any other data storage medium.

In a further embodiment of the invention, the representation of the position, as defined by the projection images, of the medical instrument in the three-dimensional structural image is not a representation in the form of a visual display, but comprises provision of the position in the data record of the three-dimensional structural image. In this case, the position information may be provided directly or else is first of all stored in a data memory, from which it can then be read again.

In a further embodiment of the invention, the information provided in this way for finding the position of the medical instrument is used for automatic guidance of the medical instrument using a planned approach route to a destination point. This allows automatic position monitoring and possibly automatic correction of the instrument guidance. At the same time, of course, it is also possible to display the position on a screen or the like. In addition to a data transmission unit for reception of the position information from the data processing unit, a corresponding apparatus comprises, inter alia, control modules for controlling robot elements or the like, and drive models for driving the robot elements.

In a further embodiment of the invention, projection images are used to detect any movement of the object. In this case, immediately successive projection recordings, that is to say in particular two or more projection recordings made during a monitoring scan to find the position of the medical instrument on each occasion, can likewise be used in the same way as projection recordings separated by greater time intervals, for example projection images of a first monitoring scan and projection recordings of a second, later monitoring scan. In this case, a check to determine whether the object has moved is preferably carried out by means of a comparison method, in particular by subtraction of the projection recordings used.

Movement is preferably detected with the aid of appropriate computer program instructions in a computer program, which is designed to be executed in a data processing unit. If the object is a patient, movement detection can be used, for example, to identify in a simple manner any movement of high-contrast structures (ribs, the spine, the instrument, etc.) as well as movements in the outer contour of the patient. It is also possible to couple movement detection to other system and method components, such as image-to-patient registration or automatic guidance of the medical instrument.

In a further embodiment of the invention, the X-ray appliance is designed to adapt the X-ray radiation while the projection images are being created. In particular, this comprises adaptation of the beam field such that the radiation dose given to the object while the projection recordings are being made is minimal. For this purpose, the X-ray appliance preferably has a drive device to drive the X-ray radiation sources to minimize the radiation dose while the projection recordings are being made.

In principle, the present invention can be combined with a multiplicity of further improvements. In particular, it is possible to couple the present invention to computer-aided navigation systems such that the navigation system records the position data of the medical instrument as input data, and uses this for navigation.

It is very particularly advantageous for the representation of the position of the medical instrument in the three-dimensional structural image to be produced in real time. The invention therefore in particular relates to a system and a method for real-time tracking of a medical instrument which is being moved in an object.

Real-time tracking such as this means a system and/or a method in which the position of the instrument is indicated within a previously fixed defined time interval, that is to say before a specific time limit is reached. In this case, however, there is no need to necessarily comply with “stringent” real-time requirements, that is to say strict preset times. Position information provided with a delay can also still be used by the user. In other words, this therefore preferably relates to a real-time method with “soft” real-time requirements.

Exemplary embodiments of the invention will be explained in more detail in the following text with reference to the drawings, in which in this case:

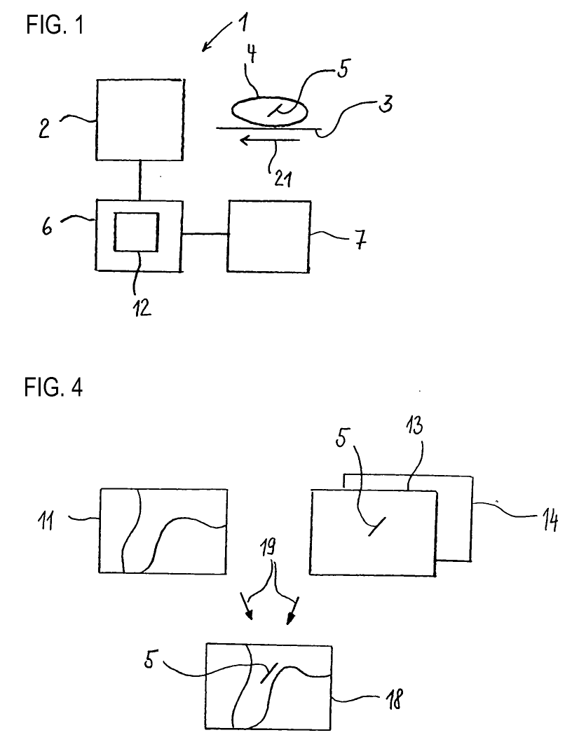

FIG. 1 shows a block diagram of a system according to the invention,

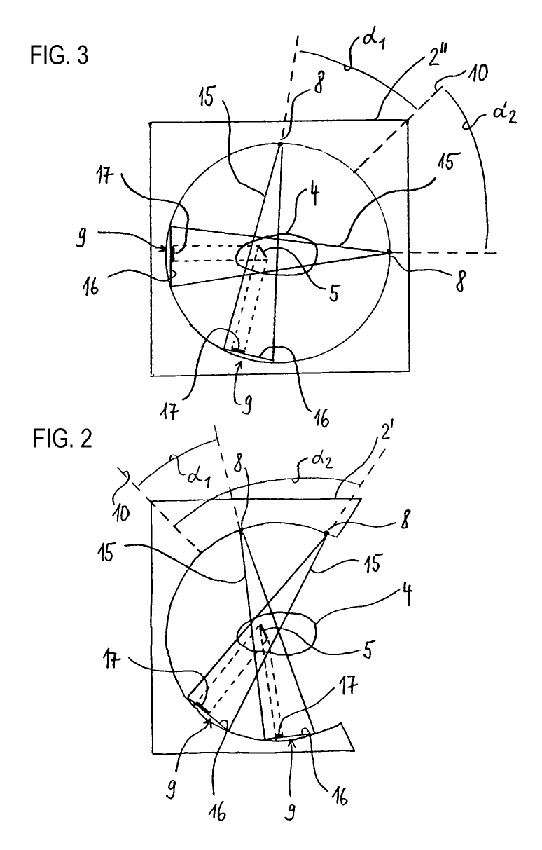

FIG. 2 shows an illustration of a C-arc X-ray appliance for use in the system according to the invention,

FIG. 3 shows an illustration of a CT X-ray appliance for use in the system according to the invention, and

FIG. 4 shows an illustration of the various types of image (structural image, projection images, result image).

All the figures illustrate the invention only schematically, and with its major components. For example, the power supply units, drive systems, stands and the like are not shown in detail.

FIG. 1 illustrates the structure of a system 1 according to the invention for medical navigation assisted by X-ray imaging. The system 1 comprises an X-ray appliance 2 for creation of a three-dimensional structural image of an object, in particular of a patient 4 lying on a patient table 3, and for creation of at least two projection images of a medical instrument 5 within the object 3, from different angles. Furthermore, the system 1 has a data processing unit 6, which is connected via a data line to the X-ray appliance 2 and to which a display appliance 7 is connected. The data processing unit 6 is in the form of the standard control unit for the X-ray appliance 2, which has been modified by having an appropriate computer program 12 added to it. A touch-sensitive screen (touch screen) is used as the display unit 7, and at the same time acts as the user interface for controlling the control unit.

The data processing unit 6 and the display unit 7 are used to display the position, as defined by the projection images, of the medical instrument 5 in the three-dimensional structural image.

In one exemplary embodiment, the X-ray appliance 2 used is a C-arc X-ray appliance 2′ with two X-ray tubes 8 and two X-ray detectors 9 in a biplanar arrangement, see FIG. 2. In this case, the nature of the X-ray tubes 8 and detectors 9 is of only secondary importance to the invention.

The procedure is as follows: the C-arc X-ray appliance 2′ is first of all used in a manner known per se to create a three-dimensional structural image of the patient 4. For this purpose, the X-ray tubes 8 and detectors 9 move about the rotation axis, generally about the longitudinal axis of the patient table 3, in a corresponding manner to the design of the C-arc. As an alternative to this, however, angled slices may, of course, also be recorded. The structural image 11 shows the anatomy of the patient 4, for example of his internal organs, see FIG. 4. The C-arc X-ray appliance 2′ is operated in accordance with the described procedure by means of the control unit, which is in the form of the data processing unit 6, with the aid of a computer program, in a manner known per se.

During subsequent transcutaneous interventions, which a doctor carries out on the basis of the structural image 11, further X-ray image recordings are created with the aid of the C-arc X-ray appliance 2′, depending on the requirement, for monitoring purposes. For this purpose, two projection images 13, 14 are in each case recorded from different angles α₁, α₂, with the patient 4 preferably remaining in the same position. By way of example, these may comprise an anterior-posterior and a lateral recording. The angles α₁, α₂ are illustrated in a simplified form relative to an initial position 10 in the figures. The two projection images 13, 14 are recorded automatically, and in each case simultaneously, with the aid of the two X-ray tubes 8 and detectors 9. Each X-ray tube 8 in this case in each case produces one X-ray beam 14, which passes through the patient 4. A shadow image is in each case created on the detector plane 16 of the X-ray detector 9 as the result of the absorption of the X-ray beams by the body of the patient 4.

Alternatively, the two or more projection recordings on a C-arc X-ray appliance can be recorded using only a single X-ray tube (not illustrated here) during rotation, with a very short time interval between them. A single-tube C-arc X-ray appliance such as this is in this case preferably designed such that the interval between two individual recordings is less than one second.

The projection images 13, 14 each show only one part of the patient 4. In this case, a patient area in which the medical instrument 5 whose position is to be found is located is selected by the doctor or automatically by the control unit. The C-arc X-ray appliance 2′ is operated, in particular with regard to the selection of the angles from which the projection images 13, 14 will be recorded, once again by the control unit that is formed by the data processing unit 6, with the aid of a software functional module according to the invention in the computer program 12.

The image 17 of the instrument 5 as projected onto the detector plane 16 of the X-ray detector 9 is imaged in each projection image 13, 14, see FIG. 4. With the knowledge of and use of the angle details α₁, α₂, the data processing unit 6 automatically determines the actual position and orientation of the medical instrument 5 in the body of the patient 4 from the two projection images 13, 14 recorded from different angles, to be more precise from the electrical image signals which are produced by the X-ray detector 9 corresponding to the projection image 13, 14 and are supplied to the data processing unit 6. For this purpose, the data processing unit 6 has a further software functional module according to the invention in the computer program 12, in which appropriate algorithms are implemented for image data processing and position calculation.

The medical instrument 5 is then automatically overlaid in the correct position and orientation on the three-dimensional structural image displayed on the display unit 7, thus creating a result image 18, see FIG. 4. The position, as defined by the projection images 13, 14, of the medical instrument 5 is superimposed on the three-dimensional structural image 11. This process is symbolized in FIG. 4 by the arrows 19. The doctor who is treating the patient 4 can use this direct position indication to monitor the intervention and if necessary to correct the instrument position. The overlaying process is carried out automatically and immediately after creation and evaluation of the projection images 13, 14 (in real time), with the instrument 5 preferably being displayed in the form of an instrument symbol. For this purpose as well, the data processing unit 6 has a further software functional module according to the invention. In particular, this functional module carries out the image-to-patient registration. This is done using information provided by the X-ray recordings and possibly by further previous method steps (for example optical localization of the patient, etc.). For simple registration, the position of the patient 4 is preferably fixed during the method. In particular, the patient 4 is located on a patient table 3, by means of which the patient 4 can be moved in a defined manner from a first position (intervention position) to a second position (X-ray position) and back. The movement of the patient is symbolized by the arrow 21 in FIG. 1.

All the image information and other information which is obtained during the method according to the invention can be processed directly (and optionally can then be stored) or else can first of all be stored in a data memory of the data processing unit 6, and can then be read again and processed further, in a further step.

In a further exemplary embodiment, a CT X-ray appliance 2″ with two X-ray tubes 8 and two X-ray detectors 9 in a biplanar arrangement is used as the X-ray appliance, see FIG. 3. The procedure is essentially identical to the procedure with the C-arc X-ray appliance as described in FIG. 2. If the CT X-ray appliance is a rotating single-tube CT X-ray appliance (not shown), then this is preferably likewise designed such that the interval between two individual recordings is less than one second.

### LIST OF REFERENCE SYMBOLS

1 System
2 X-ray appliance
3 Patient table
4 Patient
5 Medical instrument
6 Data processing unit
7 Display appliance
8 X-ray tube
9 X-ray detector
10 Initial position
11 Structural image
12 Computer program
13 Projection image
14 Projection image
15 X-ray beam
16 Detector plane
17 Image
18 Result image
19 Superimposition process
20 (Unused)
21 Positioning process

1-13. (canceled)

14. A system for medical navigation, comprising:
means for creating a three-dimensional structural image of an object;
means for creating at least two projection images of a medical instrument within the object from different angles; and
an apparatus for representing a position, as defined by the at least two projection images, of the medical instrument in the three-dimensional structural image.

15. The system according to claim 14, wherein said means for creating the three-dimensional structural image of the object and/or the means for creating the at least two projection images of the medical instrument within the object comprise an X-ray appliance.

16. The system according to claim 15, wherein said X-ray appliance is a C-arc X-ray appliance.

17. The system according to claim 16, wherein said C-arc X-ray appliance has at least two X-ray sources and being operable in simultaneous operation.

18. The system according to claim 15, wherein said X-ray appliance is a CT X-ray appliance.

19. The system according to claim 18, wherein said CT X-ray appliance has at least two X-ray sources and being operable in simultaneous operation.

20. The system according to claim 14, wherein further comprises an apparatus for automatic guidance of the medical instrument using a planned access route to a destination point.

21. The system according to claim 14, which comprises an apparatus for detection of a movement of the object on the basis of projection images.

22. The system according to claim 15, wherein said X-ray appliance is configured to adapt the X-ray radiation while the projection images are being created.

23. A method for medical navigation, which comprises the following method steps:
generating a three-dimensional structural image of an object;
generating at least two projection images of a medical instrument within the object from different angles; and
representing a position, as defined by the projection images, of the medical instrument in the three-dimensional structural image.

24. The method according to claim 23, which comprises representing the position of the medical instrument in the three-dimensional structural image in real time.

25. A computer program product for medical navigation, comprising:
computer program instructions for representing a position of a medical instrument in a three-dimensional structural image of an object, with a position of the medical instrument being defined by at least two projection images created from different angles,
when the computer program is executed on a computer.

26. The computer program product according to claim 25, comprising:
computer program instructions for creating the three-dimensional structural image of the object; and/or
computer program instructions for creating the at least two projection images of the medical instrument within the object from different angles,
when the computer program is executed on a computer.
